(19) United States
(12) Patent Application Publication
(10) Pub. No.: US 2020/0232915 A1
(43) Pub. Date: Jul. 23, 2020

(54) SPECKLE BASED SENSING OF ACOUSTIC EXCITATION IN SOLUTIONS

(71) Applicant: ContinUse Biometrics Ltd., Tel Aviv (IL)

(72) Inventors: Zeev ZALEVSKY, Rosh HaAyin (IL); Hadar GENISH, Petah-Tikva (IL)

(21) Appl. No.: 16/744,450

(22) Filed: Jan. 16, 2020

Related U.S. Application Data
(60) Provisional application No. 62/795,193, filed on Jan. 22, 2019.

(51) Int. Cl. G01N 21/49; G06K 9/00; G01N 29/02; G01N 21/47; G01N 29/24; G01N 33/49

(52) U.S. Cl. CPC G01N 21/49; G06K 9/00134; G01N 29/02; G01N 2021/479; G01N 29/2418; G01N 33/49; G06K 2209/05; G01N 21/4788

(57) ABSTRACT

The present invention relates to a system and method for determining material composition of a liquid solution. The technique includes providing acoustic excitation to the liquid solution and directing coherent illumination passing through the solution, collecting light passing through the solution by a camera unit for generating at least one image data indicative of speckle patterns. Utilizing data on variations of speckle patterns in response to excitation frequency determines material composition of the solution.

### TECHNOLOGICAL FIELD

The present invention relates to a system and method for use in determining one or more biological parameters of a subject. The technique of the invention is especially relevant for measurement of biological parameters in a liquid solution.

### BACKGROUND

Near infrared spectroscopy (NIRS) is a well-established non-invasive technique which allows for the determination of tissue and blood analyte conditions based on spectro-photometric measurements in the visible and near-infrared regions of the spectrum of light. According to this technique, incident light penetrates the examined skin, and reflected and/or transmitted light is/are measured. In order to quantify any blood analyte, light of at least two different wavelengths is required. Optical plethysmography, pulse oximetry, and occlusion spectroscopy are the most prominent examples of usage of MR spectroscopy in medicine and physiological studies.

Dynamic light scattering (DLS) is a well-established technique to provide data on the size and shape of particles from temporal speckle analysis. When a coherent light beam (laser beam, for example) is incident on a scattering (rough) surface, a time-dependent fluctuation in the scattering property of the surface, and thus in the scattering intensity (transmission and/or reflection) from the surface, is observed. These fluctuations are due to the fact that the particles are undergoing Brownian or regular flow motion and so the distance between the particles is constantly changing with time. This scattered light then undergoes either constructive or destructive interference by the surrounding particles and, within this intensity, fluctuation information is contained about the time scale of movement of the particles. The scattered light is in the form of a speckles pattern, being detected in the far diffraction zone. The laser speckle is an interference pattern produced by the light reflected or scattered from different parts of an illuminated surface. When an area is illuminated by laser light and is imaged onto a camera, a granular or speckle pattern is produced. If the scattered particles are moving, a time-varying speckle pattern is generated at each pixel in the image. The intensity variations of this pattern contain infomiation about the scattered particles. The detected signal is amplified and digitized for further analysis by using the autocorrelation function (ACF) technique. The technique is applicable either by heterodyne or by a homodyne DLS setup.

### GENERAL DESCRIPTION

The invention relates to a system and method for determining material composition of a liquid solution. The technique includes providing (acoustic) excitation to the liquid solution and directing coherent illumination passing through the solution, collecting light passing through the solution by a camera unit for generating at least one image data indicative of speckle patterns. Utilizing data on variations of speckle patterns in response to excitation frequency (generally in accordance with suitable database) determines material composition of the solution.

Therefore, according to a broad aspect of the present invention, there is provided a system for determining a material composition of a liquid solution. The system comprises an illumination unit configured and operable to generate and direct a coherent illumination beam propagating along an optical path towards the liquid solution, a vibration generator configured and operable to generate an acoustic radiation of a certain frequency range towards the liquid solution, a detector configured and operable to collect a light beam propagating along the optical path from the liquid solution being illuminated and acoustically excited and generating an image data thereof being indicative of a plurality of speckle patterns, a control unit configured and operable to control concurrent operation of the light source, the vibration generator and the detector. The control unit comprises a processing utility configured and operable to receive the image data and determine at least one of presence and concentration of at least one substance of a material composition of the liquid solution upon analyzing variation of speckle patterns.

By analyzing properties such as speckle size, contrast and/or correlation between images, it is possible to extract a signal which is proportional to the amplitude of the acoustic excitation. In some embodiments, the processing utility is thus configured for analyzing the variation of speckle patterns in response to an excitation frequency being indicative of smearing of the speckle pattern including at least one of size, correlation and contrast of the speckle. The speckle pattern data may be indicative of average speckle size. The processing utility may be thus configured for processing the image data to generate an average speckle size. The processing utility may correlate between the variation in speckle patterns in response to excitation frequency and a specific substance.

The invention is thus suitable for determining any material concentration such as glucose concentration as well as salt concentration in solutions. The processing utility may be configured for determining at least one of glucose concentration and salt concentration.

In some embodiments, the control unit is configured and operable to synchronize operation of the detector and the vibration generator such that the detector collects the light beam during an exposure time including one or more cycles of excitation frequency of the vibration generator. The speckle pattern data may be collected at exposure time corresponding to one or more cycles of excitation frequency, thereby smearing the speckle pattern in accordance with the excitation of the solution. The optical activity of glucose in aqueous solutions offers a very high specificity in detecting the presence of glucose. A contact or contactless measurement of acoustic excitations in the solution, using analysis of speckle patterns, may be performed. Solutions containing glucose should respond differently to those where glucose is absent. To perform this measurement, acoustic waves are generated towards a solution and changes in the speckle pattern are measured. The basic concept is that while the solution is acoustically excited, the acoustic waves modulate the density of the liquid under examination. This modulation will have two effects on the speckle pattern, the first being a spatial and time-varying modulation of the effective refractive index, and the second being a spatial and time-varying modulation of the optical rotation which is induced by the presence of glucose. Both of these effects should change the speckle pattern, which, if recorded with an exposure time which is longer than the acoustic period, will be seen as a smearing of the pattern.

In some embodiments, the illumination unit comprises at least one of two orthogonal polarizers placed upstream and downstream to the liquid solution, and a diffuser placed upstream to the liquid solution.

In some embodiments, the vibration generator comprises at least one of an electrical-to-acoustic transducer and an optical-to-acoustic transducer. The electrical-to-acoustic transducer may comprise a piezoelectric element configured to generate a pressure change on the solution when an electric field is applied and/or an ultrasonic transducer being configured and operable to generate ultra-sonic pressure waves at a short distance from the solution upon application of an electric field. If a piezoelectric element is used, the vibration generator may also comprise a thermoelectric element being configured for stabilizing temperature variations of the piezoelectric element. The optical-to-acoustic transducer may comprise a photoacoustic element configured to generate a pressure change on the solution when an electric field is applied.

According to another broad aspect of the present invention, there is a provided a method for determining a material composition of a liquid solution. The method comprises generating and directing a coherent light beam along an optical path towards the liquid solution, generating an acoustic radiation of a certain frequency range towards the liquid solution, wherein the light beam and the acoustic radiation are generated concurrently, collecting a light beam propagating along the optical path passing through the liquid solution while illuminated and acoustically excited and generating image data indicative of a plurality of speckle patterns, receiving the image data, analyzing variation of speckle patterns, and determining at least one of presence and concentration of at least one substance of a material composition of the liquid solution.

In some embodiments, analyzing the variation of speckle patterns in response to an excitation frequency is indicative of smearing of the speckle pattern, and includes at least one of size, correlation and contrast of the speckle. Analyzing of the variation of speckle patterns may comprise processing the image data to generate an average speckle size and/or correlating between the variation in speckle patterns in response to excitation frequency and a specific substance. Correlating between the variation in speckle patterns and a specific substance may comprise determining at least one of glucose concentration and salt concentration.

In some embodiments, the method further comprises collecting the light beam during an exposure time including one or more cycles of excitation frequency.

In some embodiments, the method further comprises orthogonally polarizing the coherent illumination beam upstream and downstream to the liquid solution respectively along the optical path.

In some embodiments, the acoustic radiation is produced by a pressure change applied on the liquid solution by using at least one of electric-to acoustic transducer and optical-to-acoustic transducer.

In some embodiments, the method further comprises stabilizing temperature variations created by the pressure change.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic block diagram showing the main functional parts of the system of the present invention;

FIG. 1B is a schematic illustration of a specific and non-limiting example exemplifying one possible configuration of the system according to some embodiments of the present invention;

FIG. 2 is a schematic flow chart showing the main functional steps of the method of the present invention; and

FIG. 3 graphically shows results obtained by using the technique of the present invention.

### DETAILED DESCRIPTION OF EMBODIMENTS

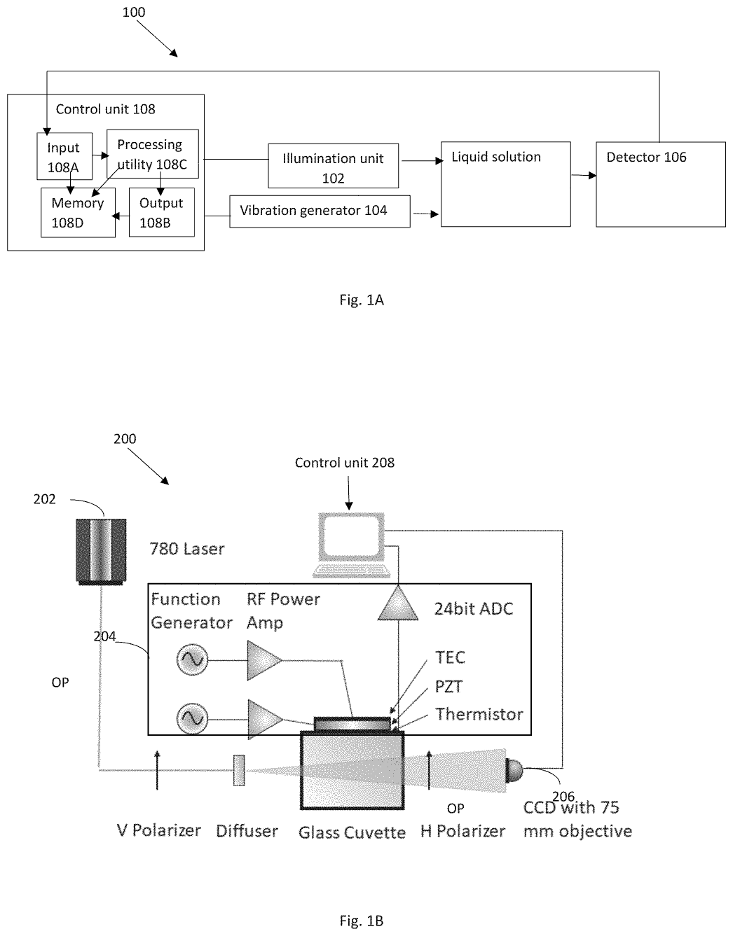

Reference is made to FIG. 1A illustrating by the way of a block diagram the main functional parts of the system 100 for determining a material composition of a liquid solution. The present invention provides a system and method which provides detection of at least one material contained in a sample such as a biological liquid (e.g. bodily liquid). The sample may be blood. System 100 comprises an illumination unit 102 configured and operable to generate and direct a coherent illumination beam propagating along an optical path towards the liquid solution, a vibration generator 104 configured and operable to generate an acoustic radiation of a certain frequency range towards the liquid solution, a detector 106 configured and operable to collect a light beam propagating along the optical path from the liquid solution being illuminated and acoustically excited and generating an image data thereof being indicative of a plurality of speckle patterns and a control unit 108 configured and operable to control concurrent operation of the light source, the vibration generator and the detector. Control unit 108 is connected to illumination unit 102, vibration generator 104 and detector 106 via wire(s) or wireless communication. Control unit 108 may comprise a switch (e.g. relay) connected via a wired or wireless connection to a remote release operating of illumination unit 102, vibration generator 104 and detector 106. Operation of illumination unit 102, vibration generator 104 and detector 106 may be synchronized. It is not shown in detail, but should be appreciated that signal exchange and communication is enabled between the units of the system by virtue of appropriate wiring, or wirelessly. For example, illumination unit 102, vibration generator 104, detector 106 and control unit 108 can be connected by IR (Infra-Red), RF (radio frequency including Bluetooth) or cable control. Control unit 108 synchronizes the operations of illumination unit 102, vibration generator 104, detector 106 and control unit 108. However, such synchronization may not be necessary since some embodiments of the present invention contemplate continuous operation of illumination unit 102. Control unit 108 performs readings of signals from detector 106 synchronously with emission of light by illumination unit 102 and generation of acoustic vibrations on the sample. In particular, control unit 108 is configured and operable to synchronize operation of detector 106 and vibration generator 104 such that detector 106 collects the light beam during an exposure time including one or more cycles of excitation frequency of vibration generator 104. This type of collection enables to obtain a less noisy measurement, since an averaging of the measurements can be performed.

Control unit 108 may comprise a general-purpose computer processor, which is programmed in software to carry out the functions described hereinbelow. Unless specifically stated otherwise, as apparent from the following discussions, it is appreciated that throughout the specification discussions utilizing terms such as “controlling”, “determining”, “analyzing”, “calculating”, “processing”, “correlating” or the like, refer to the action and/or processes of a computer that manipulate and/or transform data into other data, the data represented as physical, e.g. such as electronic, quantities. Also, operations in accordance with the teachings herein may be performed by a computer specially constructed for the desired purposes or by a general purpose computer specially configured for the desired purpose by a computer program stored in a computer readable storage medium. Control unit 108 comprises a processing utility 108C i.e. signal analyzer module configured and operable for determining at least one substance parameter (i.e. at least one of presence and concentration of at least one substance) of a material composition of the liquid solution upon analyzing variation of speckle patterns, and generating data indicative thereof. Processing utility 108C comprises a computer system comprising a data processor and being a part of and connected to a computer network. Processing utility 108C measures variations in the speckle pattern in the images received from detector 106. For example, processing utility 108C is configured for determining at least one of glucose concentration and salt concentration. Control unit 108 also comprises a data input utility 108A for receiving image data from the detector 106 and a data output utility 108B for outputting data related to at least one substance parameter of a material composition of the liquid solution. Data input and output utilities 108A and 108B may be implemented as input and output communication ports for network communication as well as for transmitting operational command to the illumination, detector and vibration generator (102, 104 and 106). Control unit 108 may also include a user interface such as screen and operation buttons for communication with an operator and providing data on operation and determined measurement results.

Detector 106 is connected to control unit 108 by wire or wireless communication and is configured to capture light beam(s) emanating from the sample along the optical path and providing this image data to the processing utility 108C (via input utility 108A). Detector 106 may be placed at the focal plane of the liquid solution to obtain focused speckles. Processing utility 108C is then configured and operable to perform Laser Speckle Contrast Analysis (LASCA) i.e. computation of contrast of focused speckles. Alternatively, detector 106 may focus on a forward displaced plane, such that the image is defocused. Defocusing brings the imaged plane into the far field. Generally the different modules may be software and/or hardware modules and may be associated with one or more processors of the control unit 108.

In some embodiments, processing utility 108C is configured for analyzing the variation of speckle patterns in response to an excitation frequency being indicative of smearing of the speckle pattern including at least one of size, correlation and contrast of the speckle. More specifically, processing utility 108C is configured for processing the image data to generate an average speckle size.

Processing utility 108C may correlate between the variation in speckle patterns in response to excitation frequency and a specific substance. Processing utility 108C may query/cross-reference the processed data being indicative of variation in speckle patterns with data in a database to generate data related to at least one substance parameter of a material composition of the liquid solution. More specifically, the variation in the speckle size, with respect to the frequency of the acoustic excitation, may represent a specific signature being indicative of a specific material composition. The technique of the present invention provides a spectral signature of a speckle size being indicative of a specific material composition. The term signature refers, for instance, to the shape and amplitude value. The correlation between the variation in speckle patterns in response to excitation frequency and a specific substance may thus include the correspondence between the specific response signature and the specific material composition, and may be stored in a database. The spectral signature of the material composition is unique for the group of compositions having the same material composition (presence and concentration). The spectral signature may include data on at least a few (e.g. one or more) materials, each such material being characterized by predetermined two or more conditions/parameters, e.g. relative to the other material. These conditions/parameters may include at least relative concentration of the respective material and relative spectral response of the material (e.g. relative number of characteristic spectral peaks associated with the presence of the material relative to at least one other material). Analyzing variation of speckle patterns may comprise identifying a radiation signature of the liquid indicative of material composition of the liquid. Processing utility 108C is configured and operable to generate an output signal indicative of whether the radiation signature of the liquid satisfies a matching condition which is assigned to a specific material composition of the liquid.

Control unit 108 may comprise a memory 108D (i.e. non-volatile computer readable medium) for storing a database e.g. reference data indicative of variation in speckle patterns vs. parameters of a specific substance. Memory 1081) may be configured for one or more storage utilities. The reference data stored in the database may be used to compare at least one variation in speckle patterns with variation in speckle patterns being indicative of parameter(s) of a specific substance stored in the database. Memory 108D may be relayed via wireless or wired connection by an external unit to a central database. Processing utility 108C may record the received data, the processed data and the output data in database in memory 108D. Processing utility 108C is adapted for identifying which of the variation in speckle patterns is indicative of parameters of a specific substance and which is not. The database may be implemented with Microsoft Access, Oracle, or other suitable commercial database systems. In some embodiments the system 100 is configured in a cloud-based configuration and/or utilizes Internet based computing so that parts of processing utility 108C, and/or memory 108D may reside in multiple distinct geographic locations. In some embodiments, storage may be separate from the server(s) (e.g. SAN storage). If separate, the location(s) of the storage may be in one physical location or in multiple locations and connected through any type of wired or wireless communication infrastructure. The database may rely on any kind of methodology or platform for storing digital data. The database may include for example, traditional SQL databases such as Oracle and MS SQL Server, file systems, Big Data, NoSQI, in-memory database appliances, parallel computing (e.g. Hadoop clusters), etc. Memory 108D may be the storage medium of the database and may include any standard or proprietary storage medium, such as magnetic disks or tape, optical storage, semiconductor storage, etc. The database may also store data relating to a specific consumer for other purposes.

Vibration generator 104 may comprise an electrical-to-acoustic transducer or an optical-to-acoustic transducer. Vibration generator 104 may or may not be in contact with the liquid solution. For example, vibration generator 104 may be placed on the surface of a container and may change the pressure therein by application of an electric field. Alternatively, vibration generator 104 may be placed at a certain distance from the solution. For example, vibration generator 104 may comprise an ultra-sonic transmitter/transducer being configured and operable to generate ultra-sonic pressure waves upon application of an electric field. Vibration generator 104 may also apply pressure changes on the solution by using the photoacoustic effect in which sound waves are formed following light absorption in a material sample. To this end, vibration generator 104 may comprise a photoacoustic element i.e. optical-to-acoustic transducer being made of a photoacoustic material. Examples of photoacoustic material include, but are not limited to, pigmented polydimethylsiloxane (PDMS), such as a mixture of PDMS, carbon black, and toluene, graphite. The photoacoustic materials may absorb the light from the illumination unit or may be coupled to another light source, or the photoacoustic material may be supplemented with dyes, e.g., organic dyes, or nanomaterials (e.g., quantum dots) that absorb the light strongly. FIG. 1B schematically shows a specific and non-limiting example of a specific configuration of the system 200 of the present invention. System 200 comprises an illumination unit configured and operable to generate and direct a coherent illumination beam propagation along an optical path OP towards the liquid solution contained in a glass cuvette, a vibration generator 204 configured and operable to generate an acoustic radiation of a certain frequency range towards the liquid solution, a detector 206 configured and operable to collect a light beam propagation along the optical path OP from the liquid solution being illuminated and acoustically excited and generating an image data thereof being indicative of a plurality of speckle patterns and a control unit 208 configured and operable to control concurrent operation of the light source, the vibration generator and the detector. Vibration generator 204 is controllable to apply changes in pressure to the liquid solution. Vibration generator 204 may or may not be in contact with the liquid solution (e.g. via a container),

In this specific and non-limiting example, vibration generator 204 comprises a piezoelectric element noted in the figure as PZT configured to generate a pressure change when an electric field is applied in accordance with an embodiment of the invention. Various piezoelectric materials that may be used in vibration generator 204 include by way of example, a piezoelectric polymer such as Polyvinylidene fluoride (PVDF), and any of various ceramics such as Lead zirconate titanate (PZT), lanthanum doped Lead zirconate titanate (PLZT), Barium titanate (BaTi03), or Sodium potassium niobate (NaKNb). In this example, vibration generator 204 comprises a function generator configured for applying a voltage to the piezoelectric material causing the piezoelectric material to contract or expand and generate thereby a change of pressure on the glass cuvette containing the liquid solution. In this specific and non-limiting example, to drive the piezoelectric element PZT, a function generator amplified with an RF power amplifier was used. The change in pressure creates an acoustic radiation propagating in the liquid solution. In this specific and non-limiting example, the liquid in the glass cuvette was excited acoustically with a piezo ceramic plate (45×45 mm plate) of Steminc™. Since the resonance frequency of the piezoelectric element PZT was found to be extremely sensitive to temperature variation, the temperature of the piezoelectric element PZT was stabilized using a thermoelectric element (e.g. thermoelectric cooler TEC). The TEC was driven by 12V applied through an H-bridge (L298N). The average current to the TEC was tuned by switching the H-bridge on and off with another function generator and controlling its duty cycle. The PZT temperature was measured with a 10K thermistor, sampled with a 24 bit analog to digital converter (LTC2400).

Illumination unit comprises a coherent light source 202 (e.g. a 780 nm laser source) configured to emit light in one or more predetermined wavelength ranges. Illumination unit may be configured to provide illumination with specific selected polarization state and orientation. For example, illumination unit may be configured to provide light with linear or elliptical polarization at a desired angle with respect to surface of the inspection region, circular polarization or random polarization, and may also include an optical arrangement enabling direction of the illumination. In this specific and non-limiting example, illumination unit comprises two orthogonal polarizers placed upstream and downstream to the liquid solution and a diffuser placed upstream to the liquid solution. The optical diffuser is configured for scattering the light beam to generate speckles. The polarizers separate between specular reflection and no specular reflection, i.e. the light beam that is not scattered emanating from the solution. More specifically, the polarization state of the non-scattered light beam is not affected by its interaction with the sample and is therefore attenuated by the cross polarizers (orthogonal polarizers). The non-scattered light beam will therefore not be collected by detector 206. The scattered light is not attenuated, as the interaction with the solution changes its polarization state and therefore will be collected by detector 206. The illumination beam passes through a vertical polarizer and is then diffused into a glass cuvette filled with a solution. In this connection, it should be noted that the glucose molecules affect polarization of returning light, and therefore this experimental setup, in which two orthogonal polarizers are placed upstream and downstream to the liquid solution, enables to determine glucose level measurement.

Detector 206 is configured for collecting light from the sample and generating an image data associated with primary speckle patterns formed by light interference via the diffuser. The speckle pattern from the diffused light was recorded using a CCD (Basler) or a high-speed camera (Fastcam, Photron). The image data collection scheme of the present technique enables collection of at least one image data within a selected temporal period from the start point of the acoustic vibration. The selected temporal period for collection of image data, within the acoustic vibration generation time, may be determined in accordance with rise time of acoustic vibrations resulting directly from the vibration generator 204.

As indicated above, control unit 108 may generally be configured as a computing unit, or as an electronic control unit, and include an operation timing module configured and operable for appropriately timing operation of the vibration generator 204, illumination unit and detector 206 in accordance with the selected operational scheme. Although not shown, control unit 108 may include an operation timing module (e.g. internal clock and timer module) configured and operable for timing operation of the different units of the system 200. The operation timing module may be connected to a vibration generator operator, an illumination unit operator and a detector operator and is configured for timing operation of the units by transmifting corresponding signals via. the respective operator modules. The operation timing module is configured to transmit operation signals to the vibration generator 204, e.g. via a vibration generator operator, for providing acoustic vibrations for a selected time period. Within the selected time period, the operation timing module operates the illumination unit operator to transmit operation signals to the illumination unit for generating at least one illumination beam, and operates the detector operator to command the detector 206 for collecting at least one image data. As indicated above, the illumination unit operator may be configured to cause the illumination unit to provide a single pulse of illumination together with acoustic vibrations, associated with operation of the function generator, while the detector 206 is operated for collecting at least one image data within duration of the illumination. Alternatively, the illumination unit operator may be configured to operate the illumination unit to provide a series of at least one pulse starting with operation of the vibration generator 204, while the detector 206 is operated, by the detector operator, for collecting at least one image data associated with the illumination pulses. It should be noted that the use of pulsed illumination of selectively short pulses enables collecting image data associated with a short time window (shorter than integration time of the PDA of the detector), such that the detector may be configured with integration time that is longer than duration of the illumination pulses.

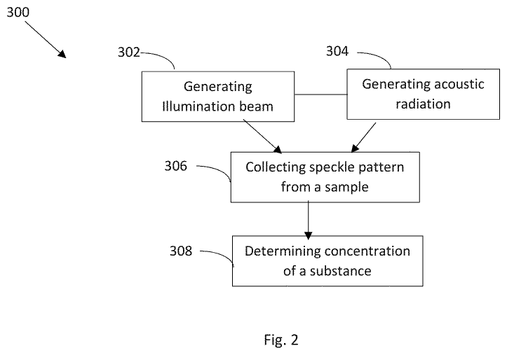

Reference is made to FIG. 2 illustrating by the way of a block diagram the main functional steps of the method 300 for determining a material composition of a liquid solution. The method 300 comprises generating and directing a coherent light beam towards the liquid solution in 302, and concurrently generating an acoustic radiation of a certain frequency range towards the liquid solution 304, collecting a light beam passing through the liquid solution while illuminated and acoustically excited, and generating image data indicative of a plurality of speckle patterns in 306, receiving the image data, analyzing variation of speckle patterns and determining at least one of presence and concentration of at least one substance of a material composition of the liquid solution in 308. Analyzing the variation of speckle patterns in 308 in response to an excitation frequency is indicative of smearing of the speckle pattern including at least one of size, correlation and contrast of the speckle. In some embodiments, analyzing of the variation of speckle patterns in 308 comprises processing the image data to generate an average speckle size and correlating between the variation in speckle patterns in response to excitation frequency and a specific substance, including determining at least one of glucose concentration and salt concentration. Collecting the light beam passing through the liquid solution in 306 may be performed during an exposure time including one or more cycles of excitation frequency. Generating and directing a coherent light beam towards the liquid solution in 302 may comprise orthogonally polarizing the coherent illumination beam upstream and downstream to the liquid solution respectively. In some embodiments, generating the acoustic radiation in 304 comprises applying a pressure change on the liquid solution. In some embodiments, the method 300 further comprises stabilizing temperature variations created by the pressure change as described above.

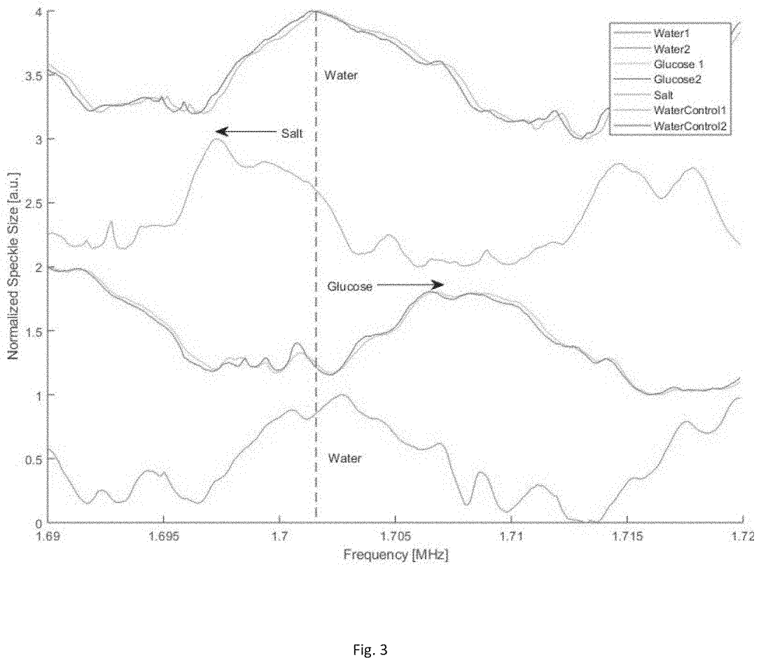

The result of the experiment described above are summarized in FIG. 3. The graph shows the normalized speckle size as a function of the frequency in MHz for different liquids. More specifically, the average speckle size was calculated from the recorded images at various frequencies of the acoustic excitation for deionized (DI) water, salt solution (1 g/DL) and for glucose solution (1 g/DL).

Each of the curves indicates the variation in the speckle size with respect to the frequency of the acoustic excitation and represent a specific signature being indicative of a specific material composition. The curves were vertically shifted for clarity. It is clear from the results that the responsivity curve of the speckle size to an acoustic excitation, which was recorded through a cuvette filled with liquid, is sensitive to the presence of substances in that liquid. The presence of these substances changes the mechanical properties of the liquid, shifts the resonance frequency of the piezoelectric material, and changes the efficiency at which the acoustic waves excite the liquid at each given frequency. The correspondence between the specific response signature and the specific material composition may be stored in a database. For example, method 300 may include storing in the database preselected data indicative of the signature of the composition. As can be seen in the figure (for water, and for glucose), the responsivity curves overlap one another. While the technique presented here is not fully contactless, it is possible to remotely sense these minute changes in the composition of solutions. The variation in the resonance frequency of acoustic sources which can excite acoustic waves can be measured by exciting a liquid from a short distance by projection of ultra-sonic waves or as e.g. occurs in the case of photo-acoustic sensing.

Between each swap of the solution, the cuvette was rinsed with DI water. As a control experiment, the cuvette was filled with DI water once again and, as can be seen, a very similar curve was obtained for responsivity of the speckle size to the acoustic excitation. The responsivity curve was sensitive to the temperature of the piezoelectric material as described above, and to avoid this effect the temperature of the piezoelectric material was stabilized until the temperature fluctuations were smaller than 0.01 degrees Celsius. To verify that the temperature was sufficiently stable, several consecutive measurements were performed at time intervals of about 5 minutes.

1. A system for determining a material composition of a liquid solution; said system comprising :
an illumination unit configured and operable to generate and direct a coherent illumination beam propagating along an optical path towards the liquid solution;
a vibration generator configured and operable to generate an acoustic radiation of a certain frequency range towards the liquid solution;
a detector configured and operable to collect a light beam propagating along the optical path from the liquid solution being illuminated and acoustically excited and generating an image data thereof being indicative of a plurality of speckle patterns;
a control unit configured and operable to control concurrent operation of said light source, said vibration generator and said detector; said control unit comprises a processing utility configured and operable to receive said image data and determine at least one substance parameter of a material composition of the liquid solution upon is analyzing variation of speckle patterns.

2. The system of claim 1, wherein said processing utility is configured for analyzing the variation of speckle patterns in response to an excitation frequency being indicative of smearing of the speckle pattern including at least one of size, correlation or contrast of the speckle.

3. The system of claim 2, wherein said processing utility is configured for processing the image data to generate an average speckle size.

4. The system of claim 1, wherein said processing utility correlates between the variation in speckle patterns in response to excitation frequency and a specific substance.

5. The system of claim 4, wherein said processing utility is configured for determining at least one of glucose concentration or salt concentration.

6. The system of claim 1, wherein said control unit is configured and operable to synchronize operation of said detector and said vibration generator such that said detector collects the light beam during an exposure time including one or more cycles of excitation frequency of the vibration generator.

7. The system of claim 1, wherein said illumination unit comprises at least one of two orthogonal polarizers placed upstream and downstream to the liquid solution or a diffuser placed upstream to the liquid solution.

8. The system of claim 1, wherein said vibration generator comprises at least one of electrical-to-acoustic transducer or an optical-to-acoustic transducer.

9. The system of claim 8, wherein said electrical-to-acoustic transducer comprises at least one of a piezoelectric element configured to generate a pressure change on the solution when an electric field is applied or an ultrasonic transducer.

10. The system of claim 9, wherein said vibration generator comprises a thermoelectric element being configured for stabilizing temperature variations of said piezoelectric element.

11. The system of claim 8, wherein said optical-to-acoustic transducer comprises at least one of a photoacoustic element configured to generate a pressure change on the solution when an electric field is applied.

12. A method for determining a material composition of a liquid solution; said method comprising:
generating and directing a coherent light beam along an optical path towards the liquid solution;
generating an acoustic radiation of a certain frequency range towards the liquid 20 solution; wherein said light beam and said acoustic radiation are generated concurrently;
collecting a light beam propagating along the optical path passing through the liquid solution while illuminated and acoustically excited, and generating image data indicative of a plurality of speckle patterns,
receiving said image data, analyzing variation of speckle patterns and determining at least one of presence or concentration of at least one substance of a material composition of the liquid solution.

13. The method of claim 12, wherein said analyzing of the variation of speckle patterns in response to an excitation frequency is indicative of smearing of the speckle pattern including at least one of size, correlation or contrast of the speckle.

14. The method of claim 12, wherein said analyzing of the variation of speckle patterns comprises processing the image data to generate an average speckle size.

15. The method of claim 12, wherein said analyzing of the variation of speckle patterns comprises correlating between the variation in speckle patterns in response to excitation frequency and a specific substance.

16. The method of claim 15, wherein said correlating between the variation in speckle patterns and a specific substance comprises determining at least one of glucose concentration or salt concentration.

17. The method of claim 12, wherein said collecting the light beam comprises collecting the light beam during an exposure time including one or more cycles of excitation frequency.

18. The method of claim 12, wherein said generating and directing of a coherent light beam comprises orthogonally polarizing said coherent illumination beam upstream and downstream to the liquid solution respectively along the optical path.

19. The method of claim 12, wherein said generating the acoustic radiation comprises applying a pressure change on the liquid solution by using at least one of electric-to-acoustic transducer or optical-to-acoustic transducer.

20. The method of claim 19, further comprising stabilizing temperature variations created by said pressure change.
